(19) United States
(12) Patent Application Publication
(10) Pub. No.: US 2002/0161332 A1
(43) Pub. Date: Oct. 31, 2002

(54) Infusion set with tape

(76) Inventor: Kirk Ramey, Bedford, VA (US)

Correspondence Address: ANN M. SKERRY, Esq., Fay, Sharpe, Fagan,, Minnich & McKee, LLP, 1100 Superior Avenue, 7th Floor, Cleveland, OH, 44114-2518, US

(21) Appl. No.: 10/122,020

(22) Filed: Apr. 12, 2002

Related U.S. Application Data
(60) Provisional application No. 60/283,575, filed on Apr. 13, 2001.
(60) Provisional application No. 60/285,371, filed on Apr. 20, 2001.

(51) Int. Cl. A61M 5/178

(52) U.S. Cl. 604/164.07; 604/180

(57) ABSTRACT

An infusion set (1, 100, 100′) is attached to the skin of a wearer by a conformable tape or patch (220, 220′). The tape is of a multilayer construction, comprising a breathable layer (226, 226′), preferably formed from a silicone elastomer or hydrocolloid, and optionally a support layer (222), such as a layer of polyester. An adhesive layer (240) is provided for attaching the tape to the wearer's skin. A second adhesive layer (234), or a mechanical means (252) attaches the tape to the infusion set.

This application claims the priority of U.S. Provisional Application Serial No. 60/283,575, filed Apr. 13, 2001, and U.S. Provisional Application Serial No. 60/285,371, filed Apr. 20, 2001.

### BACKGROUND OF THE INVENTION

The present invention relates to infusion systems for subcutaneous delivery of a medication or a therapeutic fluid into the body and more particularly to an infusion device for releasably coupling an infusion pump to a subcutaneous cannula for delivery of the medication or the therapeutic fluid.

Portable infusion devices are generally known in the art for delivering medications and other fluids, such as insulin, sugars, and painkillers, to a subcutaneous site in the body of a patient. Such devices commonly include a tubular cannula extending from a connector housing which receive the desired medication from an infusion delivery system, such as a syringe pump, or the like. The housing is typically in two parts, allowing the user to disconnect the cannula from the pump to facilitate activities, such as bathing, swimming, or the like, for which it is desirable to have the pump disconnected.

In use, the housing provides a fluid-tight connection between the housing and the tubular cannula that helps to prevent contamination of the injection site. For example, the housing may include a first portion, connected with the cannula, which includes a self-sealing penetrable septum, and a second, disconnectable portion, connected with the pump and having a hollow needle adapted to penetrate the septum. When the needle is withdrawn from the septum, a fluid-tight seal is created between the interior of the first portion of the housing and the cannula. The septum and the needle further provide a fluid-tight seal between the cannula and a delivery tube when the medication is being delivered to the patient from the external infusion system.

Conventional infusion sets of this type tend to be unwieldy and difficult for the user to operate. There remains a need for an infusion set which is simple to operate without injury or discomfort to the user, yet which provides assurance against accidental disconnection during use.

U.S. Pat. No. 5,522,803 shows an infusion set including a cannula housing, which interconnects with a needle holder. A feed line feeds a medicament to the needle holder. A connecting needle of the needle holder is introduced into a channel of the cannula housing via a septum. Guide pins, one on either side of the needle, are provided for correctly positioning the needle in the channel of the cannula housing. During advancement of the needle into the channel, the needle holder is anchored to the cannula housing by a pair of automatically engaging snap-fit tabs.

The present invention provides a new and improved infusion set and attachment system for adhering the set to a wearer's body which overcomes the above-referenced problems, and others.

### SUMMARY OF THE INVENTION

In accordance with one aspect of the present invention, an infusion set and attachment system combination is provided. The combination includes a conformable patch for attaching the infusion set to the skin of a wearer. The infusion set includes a first housing portion connected to a top layer of the patch. A second housing portion is selectively interconnectable with the first housing portion for forming a fluid passageway therebetween. The second housing portion is connected with a source of a liquid to be introduced to the wearer. A cannula is connected with the first housing portion for introducing the liquid to the wearer, the cannula passing through the patch when inserted into the wearer's skin.

In accordance with another aspect of the present invention, an infusion set is provided. The infusion set includes a first housing portion which is configured to be attached to a wearer's skin,. The first housing portion defines a first fluid passage. A second housing portion is selectively connectable with the first housing portion. The second housing portion defines a second fluid passage. A sealing means is biased to a sealing position to seal one of the first and second passages when the first and second housing portions are disconnected. The sealing means is moved to a non-sealing position during interconnection of the first and second housing portions such that the first and second passages to form a fluid flowpath whereby a liquid flows from the first housing portion to the second housing portion. Connection means are provided for selectively interconnecting the first and second housing portions.

In accordance with another aspect of the present invention, a method for supplying a liquid to a person is provided. The method includes attaching a patch to the skin of the user and attaching a first housing portion to the patch. A cannula of the first housing portion is inserted through the patch and into the person's skin. A second housing portion is selectively coupled with the first housing portion to form a fluid flowpath therebetween which is fluidly connected with the cannula. The liquid is fed to the first housing portion, the liquid flowing along the fluid flowpath and into the cannula.

One advantage of one embodiment of the present invention is that it is easily operated by a user.

Another advantage of one embodiment of the present invention resides in its low profile.

Another advantage of one embodiment of the present invention is that the multi-layer patch provides a support region underlying the infusion set while providing a breathable region adjacent the wearer's skin in the area of penetration of the cannula.

Another advantage of the present invention is that the infusion set is readily attached to or removed from the patch, when desired.

Still further advantages of the present invention will become apparent to those of ordinary skill in the art upon reading and understanding the following detailed description of the preferred embodiments.

### BRIEF DESCRIPTION OF THE DRAWINGS

The invention may take form in various components and arrangements of components, and in various steps and arrangements of steps. The drawings are only for purposes of illustrating a preferred embodiment and are not to be construed as limiting the invention.

FIG. 1 is a top plan view of a two part infusion system according to the present invention;

FIG. 1A is an enlarged view of the stopper and tube of FIG. 1;

FIG. 2 is a top plan view of the two part infusion system of FIG. 1 with the two parts connected;

FIG. 3 is a side sectional view of the infusion system of FIG. 1;

FIG. 3A is an enlarged view of the cannula housing showing an O-ring for attaching the cannula housing to a cavity of the patch;

FIG. 4 is a side sectional view of the infusion system of FIG. 1 showing insertion of a cannula;

FIG. 5 is a side sectional view of the infusion system of FIG. 1 after insertion of a cannula;

FIG. 6 is a top plan view of the two part infusion system of FIG. 1 showing a feed tube and Luer connector;

FIG. 7 is an exploded perspective view of an alternative embodiment of an infusion system according to the present invention;

FIG. 8 is a side sectional view of the needle housing of FIG. 7 with the needle removed;

FIG. 9 is a top sectional view of the needle housing of FIG. 7;

FIG. 10 is a cross sectional view of the needle housing of FIG. 7;

FIG. 11 is a side perspective view of the cannula housing of FIG. 7;

FIG. 12 is a side sectional view of the infusion set of FIG. 7 after assembly;

FIG. 13 is a side sectional view of an insertion set according to the present invention;

FIG. 14 is an exploded perspective view of the insertion set of FIG. 13;

FIG. 15 is a perspective view of the tip of the needle and catheter of FIG. 13;

FIG. 16 is a top plan view of the adhesive patch of FIG. 7;

FIG. 17 is a side sectional view of the adhesive patch of FIG. 16 through A-A;

FIG. 18 is a side sectional view of the adhesive patch of FIG. 16 through B-B;

FIG. 19 is an exploded side sectional view of the adhesive patch of FIG. 16 through A-A;

FIG. 20 is an exploded top view of the adhesive patch of FIG. 16;

FIG. 21 is a top plan view of an alternative embodiment of the adhesive patch of FIG. 7;

FIG. 22 is a side sectional view of the adhesive patch of FIG. 21 through A-A;

FIG. 23 is a side sectional view of the adhesive patch of FIG. 21 through B-B;

FIG. 24 is an exploded top view of the adhesive patch of FIG. 21;

FIG. 25 is a partially exploded perspective view of an alternative embodiment of an infusion set according to the present invention;

FIG. 26 is a side sectional view of the infusion set of FIG. 25;

FIG. 27 is a top view of one embodiment of the patch with an insertion set thereon; and

FIG. 28 is a side sectional view of the embodiment of FIG. 27.

### DETAILED DESCRIPTION OF THE PREFERRED EMBODIMENTS

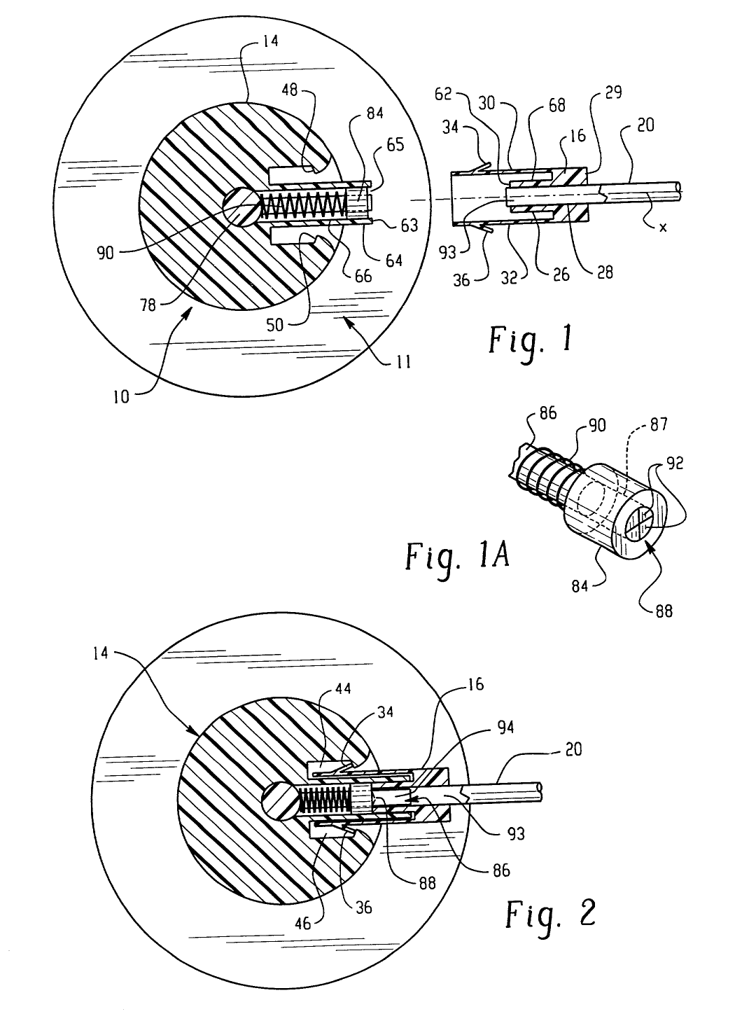

With reference to FIGS. 1-6, a first embodiment of an infusion set 1 includes a two-part assembly or housing 10 with a generally planar configuration when assembled. An attachment system 11 attaches the housing to a wearer's skin S. A soft catheter or cannula 12 extends from a first portion or cannula housing 14 of the assembly and is received through the skin S of the wearer of the infusion set. The cannula is formed from a soft plastic material and has a central bore 15 through which a medicament, such as insulin, or other liquid to be infused is supplied from the assembly 10 into the user's body. A second portion or connector housing 16 of the assembly 10 is configured for releasable interlocking with the cannula housing 14. The first portion 14 of the housing 10 is designed to remain on the user's skin for extended periods, while the second portion 16 is removed at intervals for bathing or other purposes. The connector housing 16 acts as a needleless-connector and is of a thinner cross section d than the cannula housing.

The connector housing 16 is fluidly connected with a feed tube 20. The feed tube 20 supplies the medicament to be injected from an infusion pump (not shown), or other supply source, to the connector housing 16. The feed tube 20 is fluidly connected with the infusion pump via a Luer connection 22 or other suitable releasable connection (FIG. 6). Although the system 1 is described in terms of fluid flowing downstream from the feed tube 20 to the skin via the infusion set, it is also to be appreciated that fluid may be pumped from the skin into feed tube via the infusion set (i.e., in an opposite direction), for sampling of body fluids. The two housing portions 14, 16 may be formed from plastic, or other suitable material by molding or other fabrication process.

With particular reference to FIG. 1, the connector housing 16 includes a central portion 26, which receives the feed tube 20 at least partially therethrough. Specifically, the central portion defines a bore or passage 28 with a central axis x. FIG. 1 shows the feed tube 20 extending the entire length of the bore 28, although it is to be appreciated that the feed tube may end closer to the upstream end of the bore or be connected therewith by a fitting (not shown). Two resiliently flexible arms 30, 32 extend laterally from and generally parallel with the central portion 26 and are connected at a rearward (upstream) end 29 with the central portion 26. (The terms rearward, forward, upper and lower, and the like are used herein with reference to the orientation of the infusion set components illustrated in FIG. 1). The arms 30, 32 are thus positioned 180° apart, one on either side of the central portion 26 and are aligned generally parallel therewith.

Ends of the respective flexible arms 30, 32 define resilient, outwardly projecting hooks 34, 36 for releasably locking the connector housing 16 to the cannula housing 14. The hooks project forward of the central portion 26. When a user grips the arms 30, 32 and squeezes them between his fingers, the distal ends of the arms flex inwardly, bringing the hooks 34, 36 closer together. The hooks are received in corresponding internal slots 44, 46 of the cannula housing (see FIG. 2) and engage suitably shaped and positioned shoulders 48, 50, respectively. During insertion, the hooks 40, 42 function as camming surfaces, causing the arms 30, 32 to flex inwardly and allowing the hooks 34, 36 to snap past the shoulders 48, 50, automatically locking the connector housing 16 to the cannula housing 14 as the parts 14, 16 are advanced towards each other. To release the connector housing 16 from the cannula housing 14, the arms 30, 32 are pressed inwards while the connector housing 16 is retracted from the cannula housing 14.

The connector housing 16 is axially symmetrical, allowing the connector housing to be connected with the cannula housing 14 in one of two orientations, either as shown in FIG. 1, or flipped 180°.

A forward (downstream) end 62 of the central portion 26 is selectively received within a forward (upstream) end 63 of a corresponding projection 64 on the cannula housing 14. The projection 64 is shaped for guiding receipt of at least the downstream end of the central portion. Specifically, the projection defines a bore 65, which extends axially through the projection. An interior wall 66 of the bore may be slightly tapered and matches the taper of an exterior surface 68 of the central portion 26.

As can be seen from FIG. 1, the hooks 34, 36 on the arms project forwardly of the cylindrical central portion 26 of the body portion, although it is also contemplated that the hooks may extend no further than the cylindrical central portion or be set back therefrom.

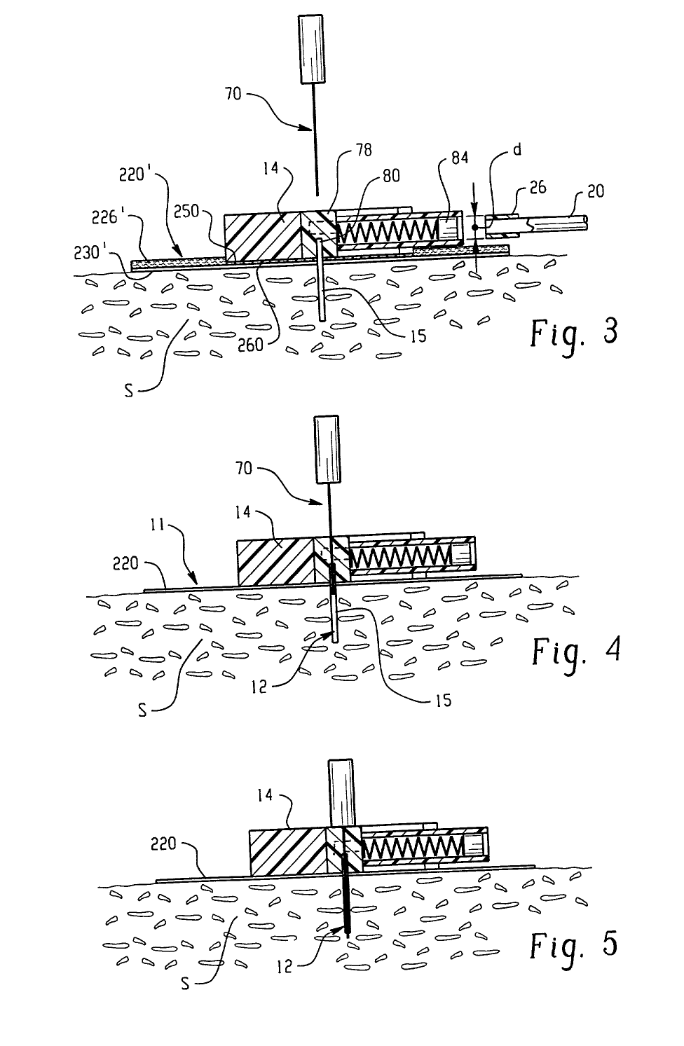

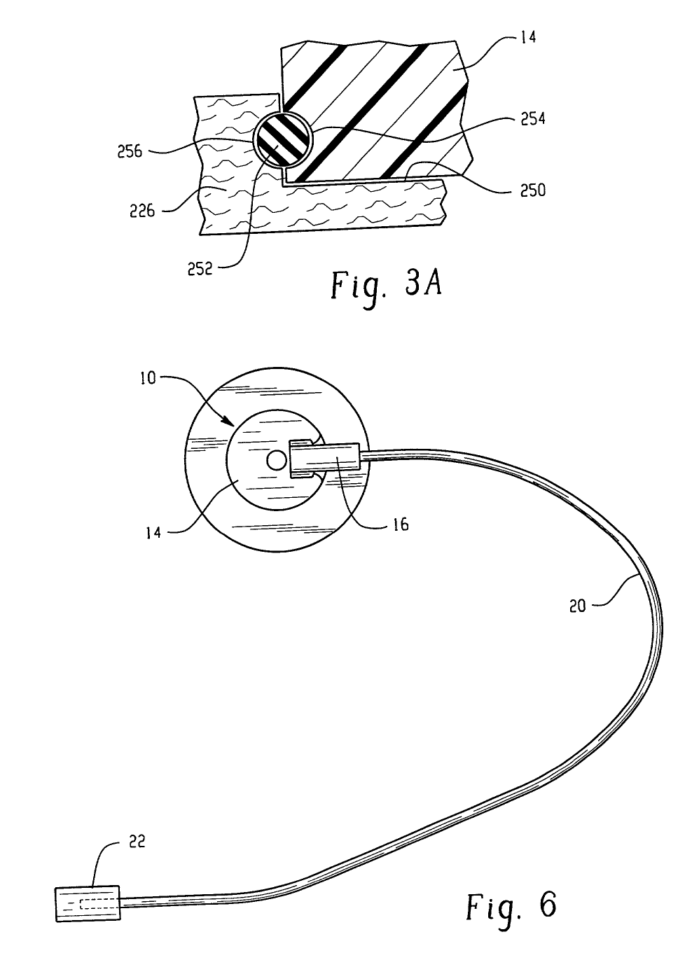

With reference to FIGS. 3-5 the cannula housing 14 receives a cannula insertion needle 70 in the form of a fine bored tube. The canula needle serves to introduce the cannula into the user's skin and is withdrawn after the catheter has been inserted. The cannula needle is selectively insertable into a seal or septum 78 in the cannula housing, such as a cylindrical, self-sealing silicone seal. The septum seals a hole formed in the catheter 15 by needle at an upper end 80 thereof. The self-sealing septum provides a liquid and air seal towards the environment when the needle 70 is retracted from the septum and further provides a liquid and air seal around the needle when inserted through the septum.

As shown in FIG. 1, the bore 65 of the cannula housing 14 extends between the forward end 63 of the projection and the septum 78.

An annular stopper 84 is received within the bore 65. The stopper encircles a tubular member, such as a tube 86, which extends axially along the bore 65 between the forward end 63 of the projection and the catheter entrance 80. The stopper defines an interior passage 87 (FIG. 1A). The stopper is formed from a resiliently flexible material and sealingly engages an exterior surface of the tube while also sliding relative thereto. The stopper preferably defines a valve 88 at a forward end thereof, such as a flap valve.

The stopper 84 is biased to the position shown in FIG. 1 by a biasing member 90, such as a spring. The spring encircles the tube 86 and is held under compression between the stopper and the septum 78. Optionally, a flange (not shown) at the forward end 63 of the bore 65 prevents the stopper 84 from exiting the bore 65. In the position shown in FIG. 1, the flap valve 88 is in a closed position, sealing the tube 86 from the atmosphere.

When the central member 26 is inserted into the bore 65, the forward end 62 of the central member engages the stopper 84 and pushes it rearwardly into the projection, against the bias of the spring, to the position shown in FIG. 2. The tube 86 opens the flap valve 88 in the process (for example, pushes apart four flexible flaps 92 which make up the valve) and enters the bore 28 of the connecting member and passes into the feed tube 20. Fluid can then flow from a first passage 93 defined by the feed tube 20 (and/or by the bore 28 if the feed tube 20 does not extend entirely along the bore 28) into an interior passage 94 of the tube 86 and thence to the catheter 15. While the tube 86 is coupling with the bore 28 to form a connecting passageway 93, 94, the hooks 34, 36 are entering the respective internal slots 44, 46 of the cannula housing and engaging the shoulders 48, 50. With the two components 14, 16 thus locked together the stopper maintains a seal between the tube 86 and the forward end 62 of the central portion (and/or the feed tube if it extends forwardly of the forward end 62) and prevents leakage of liquid from the housing 10. Alternatively, at least one of the tube 86 and the bore 28 is tapered such that the adjacent surfaces of the tube 86 and bore 28 (or feed tube 20) frictionally engage each other during coupling of the housing portions 14, 16 to provide a seal therebetween.

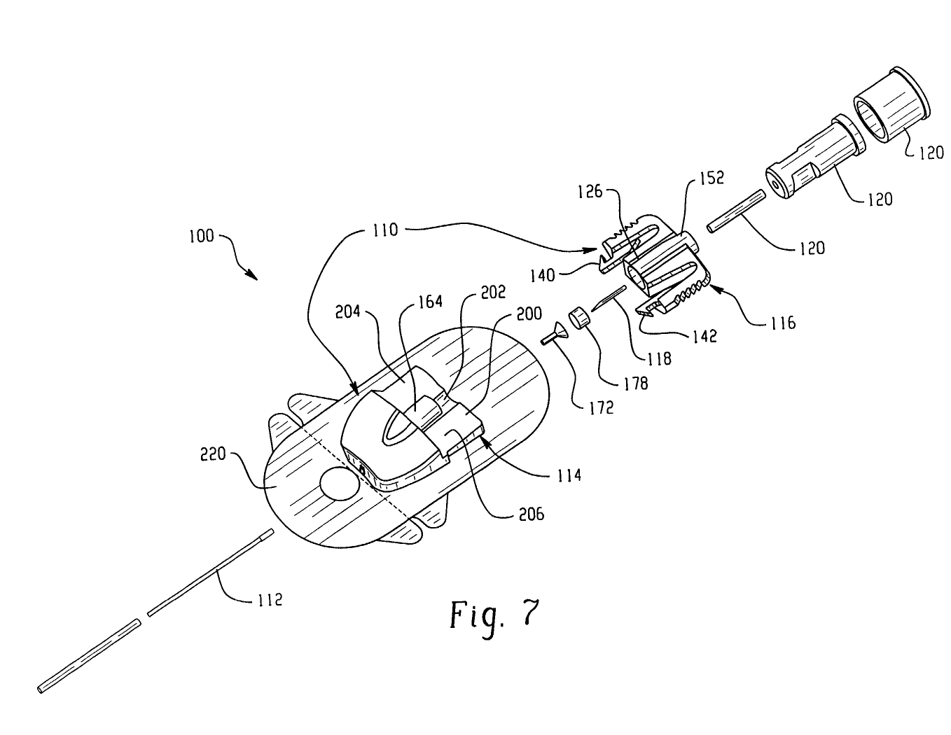

With reference to FIG. 7, an alternative embodiment of an infusion set 100 includes a two-part assembly or housing 110 with a generally planar configuration when assembled. A soft cannula or catheter 112 extends from a first portion or cannula housing 114 of the assembly and is received through the skin (not shown) of a user of the infusion set. The cannula is formed from a soft plastic material and has a central bore 115 through which a medicament, such as insulin, or other liquid to be infused is supplied from the assembly 110 into the user's body. A second, connector portion or needle housing 116 of the assembly 110 is configured for releasable interlocking with the cannula housing 114 in a similar manner to that described for the embodiment of FIGS. 1-6. The first portion 114 of the housing 110 is designed to remain on the user's skin for extended periods, while the second portion 116 is removed at intervals for bathing or other purposes. The needle housing 116 is of a thinner cross section d than the cannula housing.

The needle housing 116 carries a needle 118, which is fluidly connected with a feed tube 120 analogous to the feed tube 20. The feed tube 120 supplies the medicament to be injected from an infusion pump (not shown), or other supply source, to the needle 118. The feed tube 120 is fluidly connected with the infusion pump via a Luer connection 122 or other suitable releasable connection. The two housing portions 114, 116 may be formed from plastic, or other suitable material by molding or other fabrication process.

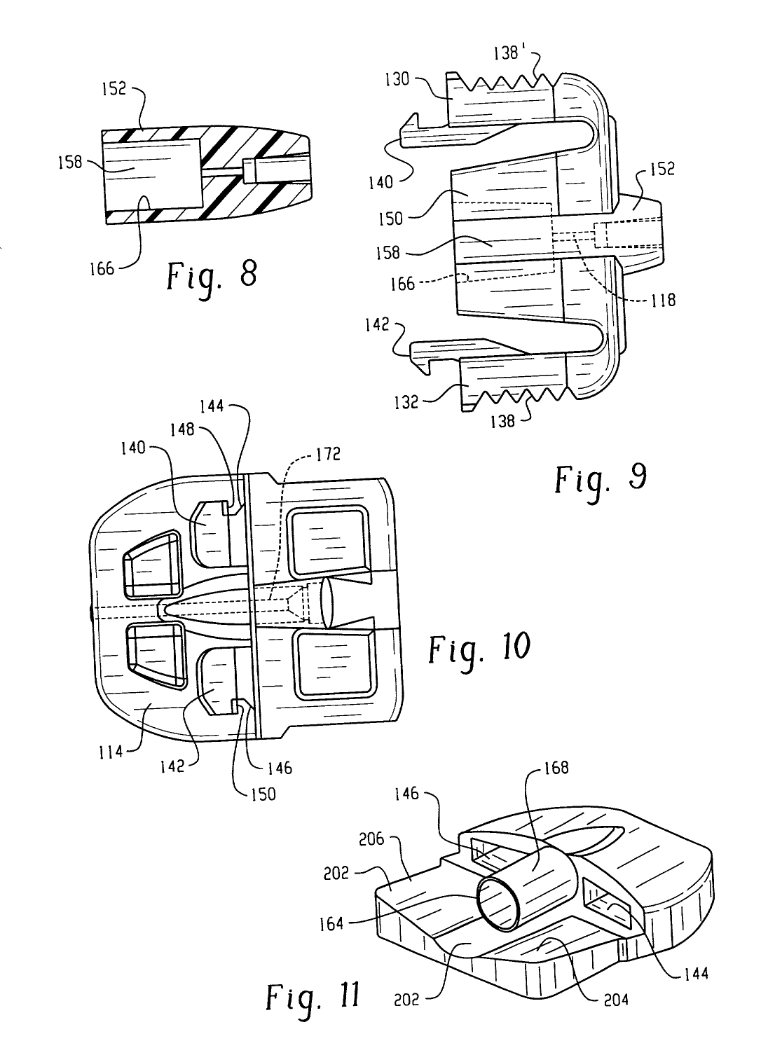

With reference also to FIGS. 8-11, the needle housing 116 includes a body portion 126, which is shaped to provide a cover for the needle 118 and to receive the feed tube. Specifically, the body portion includes a central portion 128, in the form of a generally cylindrical tube with a central axis x, and two resiliently flexible arms 130, 132, which extend laterally from and generally parallel with the central portion 128. The arms 130, 132 are connected at a rearward (upstream) end 129 of the body portion with the central portion 128. (The terms rearward, forward, upper and lower, and the like are used herein with reference to the orientation of the infusion set components illustrated in FIG. 7). The arms 130, 132 are thus positioned 180° apart, one on either side of the needle 118 and are aligned generally parallel therewith.

A pair of resilient, hook-shaped tabs 134, 136 extend from distal ends of the respective flexible arms 130, 132, on either side of the needle, for releasably locking the needle housing 116 to the cannula housing 114 and project forward of the central portion 128. The two arms 130, 132 include finger-gripping regions 138, respectively, defined by serrated edges of the arms 130, 132. When a user grips the finger-gripping regions 138 and squeezes them between his fingers, the distal ends of the arms flex inwardly, bringing the tabs 134, 136 closer together. Distal ends of the tabs 134, 136 define hooks 140, 142 having outwardly extending ends. The hooks are received in corresponding internal slots 144, 146 of the cannula housing (see FIGS. 10 and 11), and engage suitably shaped and positioned shoulders 148, 150, respectively. During insertion, the hooks 140, 142 function as camming surfaces, causing the arms 130, 132 to flex inwardly and allowing the hooks 140, 142 to snap past the shoulders 148, 150. To release the needle housing 116 from the cannula housing 114, the arms 130, 132 are pressed inwards while the needle housing 116 is retracted from the cannula housing 114.

Returning to FIGS. 7-9, the central portion 128 of the needle housing comprises an axial, generally cylindrical tube for protecting the needle 118. The tube 128 protrudes above and below the rest of the body portion such that the profile of the body portion is substantially the same on both upper and lower surfaces 154, 156 thereof. This allows the needle housing 116 to be connected with the cannula housing 114 in one of two orientations, either as shown in FIG. 7, or flipped 180°.

An axial bore 158 is formed within the tube 128, in which the needle 118 is concentrically positioned. The bore 158 has an opening 160 at an end 162 of the tube 128 facing the cannula housing 114. The tube 128 is slidingly received on a mating projection 164 on the cannula housing when the tabs 134, 136 are engaged with the respective cannula housing shoulders 148, 150. The projection 164 is shaped for guiding receipt of the tubular portion 128. Specifically, an interior wall 166 of the tubular portion bore 128 may be slightly tapered and matches the taper of an exterior surface 168 of the projection 164.

As can be seen from FIG. 7, the hooks 140, 142 on the tabs project forwardly of the cylindrical portion 128 of the body portion, although it is also contemplated that the tabs may extend no further than the cylindrical portion or be set back therefrom.

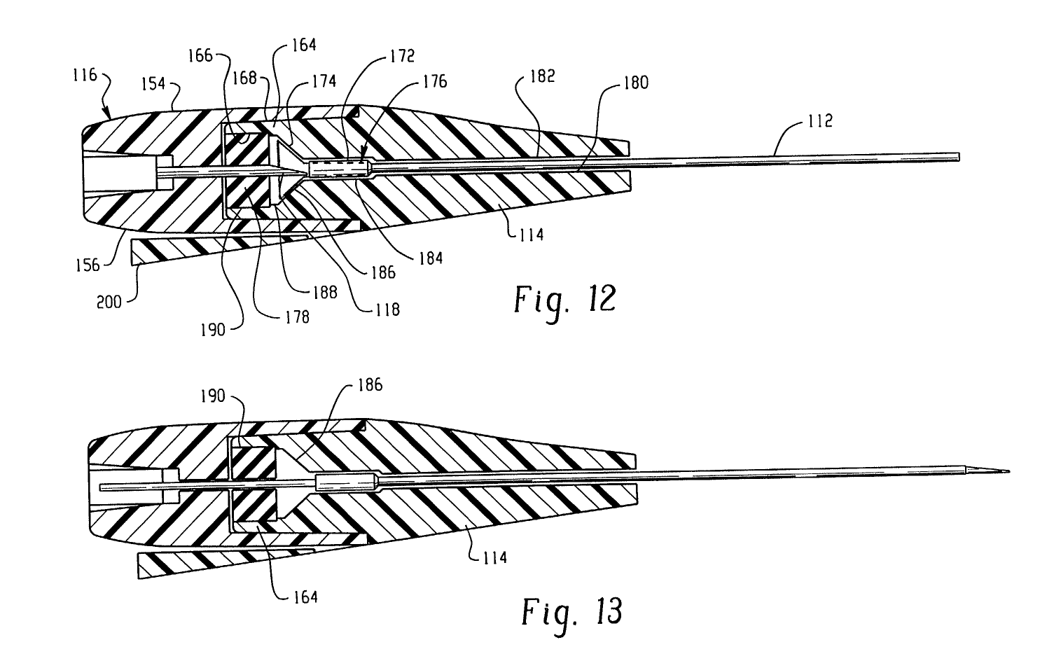

With reference to FIG. 7 and also to FIG. 12, the cannula housing projection 164 carries a needle guide 172, preferably formed from metal or alloy, such as stainless steel. The needle guide 172 includes an open funnel portion 174, which faces outwardly from the canula housing, and a tubular portion 176 connected therewith, which is fluidly connected with the cannula 112. A seal or septum 178, such as a cylindrical, self-sealing silicone seal, may be seated upstream of the funnel portion 174 of the needle guide 172, as shown in FIG. 12, or downstream of the needle guide. The self-sealing septum provides a liquid and air seal towards the environment when the needle 118 is retracted from the septum and further provides a liquid and air seal around the needle when inserted through the septum.

As shown in FIG. 12, the cannula housing 114 defines an interior bore 180 therethrough, which is shaped to house the cannula, needle guide 172 and seal 178. The cannula housing bore 180 extends axially through the projection 164. As can be seen, the bore 180 includes a series of interconnected sections of increasing internal diameters, a first narrow cylindrical section 182, which houses an upstream end 183 of the cannula, a second, slightly wider cylindrical section 184, which houses the tubular portion 176 of the needle guide, a third, tapered conical section 186, which follows the shape of the funnel portion 174 of the needle guide, a fourth cylindrical section 188 of the same diameter as the widest end of the conical section 186, and a fifth, cylindrical section 190, which is of slightly larger diameter than the fourth section. The seal 178 is shaped such that it fits snugly in the fifth section 190. The fourth section 188, being of slightly smaller diameter than the fifth section, spaces the seal from the needle guide. This section may be eliminated, if desired.

To assemble the parts of the cannula housing 114 of this embodiment, the cannula 112 and needle guide 172 are joined together and inserted into the bore 180 from the widest end. Then the seal 178 is simply press fit into the fifth section 190 of the bore. An adhesive may be applied to the edges of the seal and/or to the sides of the fifth section to secure the seal in the bore 180.

With reference to FIGS. 7 and 11, the cannula housing 114 includes a shelf 200 which extends generally parallel with a spaced below the projection 64 and defines a longitudinal concave trough 202. The trough 202 is shaped to receive the cylindrical portion 128 of the needle housing body therein. Since the body portion has essentially the same profile on both its larger surfaces (top and bottom surfaces 154, 156 in FIG. 12), the cylindrical portion enters the trough when the needle housing is connected in either of two orientations. The rest of the body portion and tabs rest on the adjacent flat upper surfaces 204, 206 of the shelf.

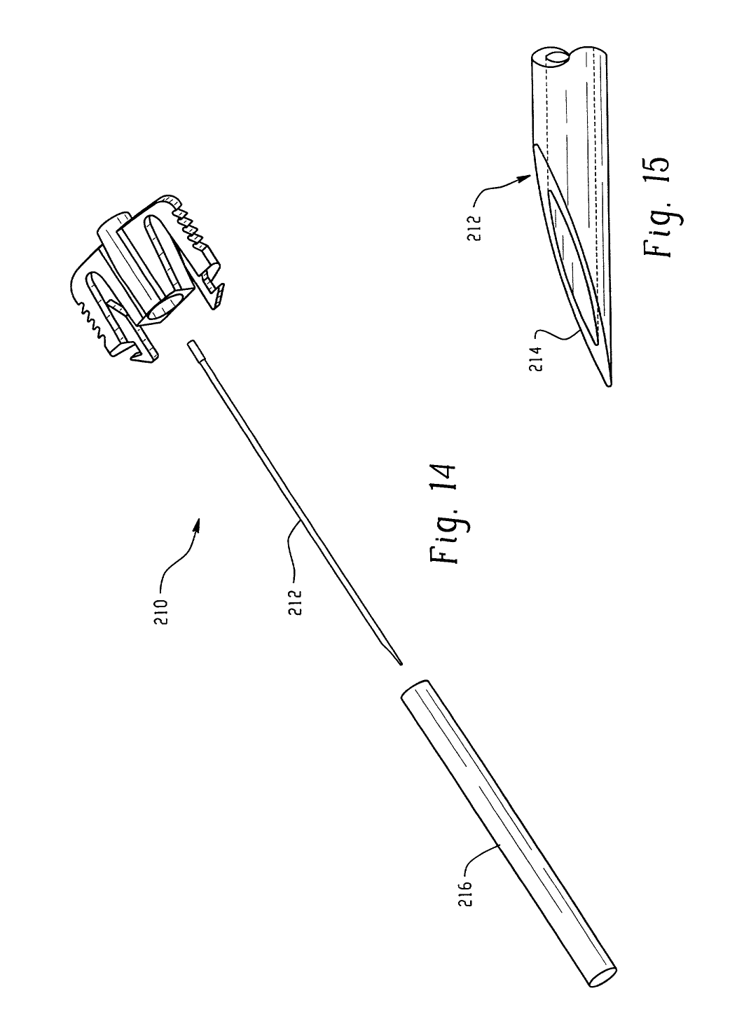

FIGS. 13 and 14 show an insertion set 210 which can be used with the cannula housing 114 to insert the cannula into the skin of the user. The insertion set has a longer needle 212 than is used with the needle housing 116, which extends to the end of or slightly beyond the distal tip 214 of the cannula when fully inserted (See FIG. 15). As can be seen from FIG. 13, the cannula may be inserted in to the skin prior to placing the septum in the cannula housing bore. This avoids potential damage to the septum by the insertion needle 214. Once the cannula has been positioned in the users body, the insertion set 210 is removed and the seal 178 inserted in the cannula housing. Or, the seal can be inserted and glued into the bore prior to insertion of the cannula into the skin for convenience. A cover 216 protects the tip of the needle 214 when not in use (FIG. 14).

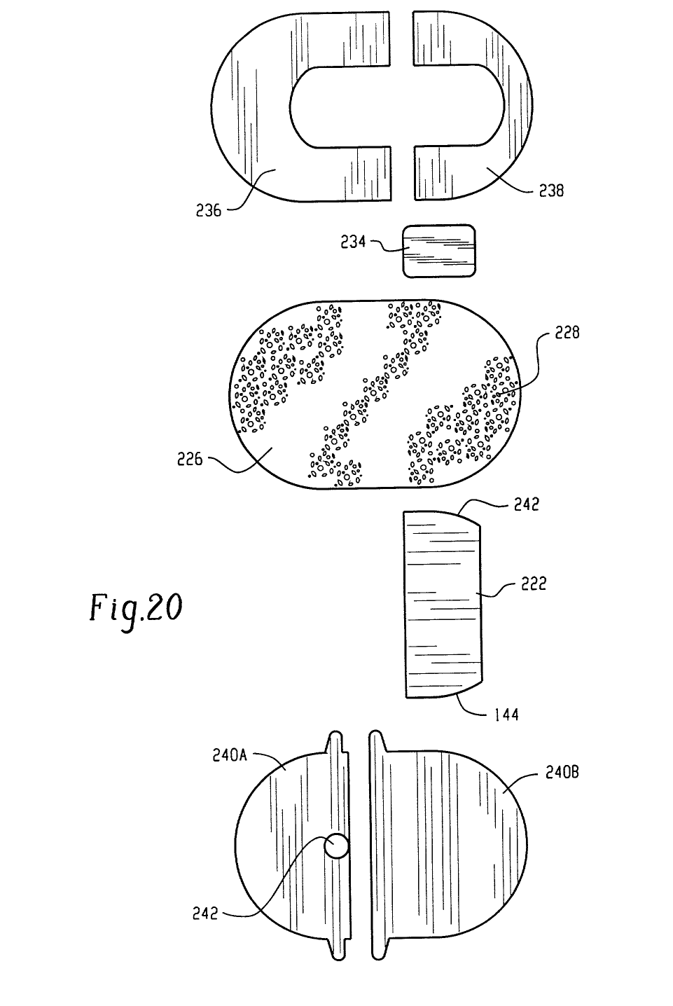

With reference to FIGS. 1-7, and 16-20, the infusion set of either embodiment is held in position by the attachment system 11, which in the preferred embodiment, comprises an adhesive patch or tape 220. Preferably, the patch is of a multi-layer construction, and includes a structural support layer 222, for providing structural support to the infusion set, such as a layer of a non-woven material, e.g., polyester. The support layer has a layer 224 of an adhesive on its lower surface (see FIG. 19), such as a pressure sensitive adhesive (PSA), e.g., an acrylic, for attaching the layer firmly to the wearer's skin. A suitable structural layer 222 is an embossed non-woven polyester fabric, about 1.5 mm in thickness, sold under the tradename Hypofix. Attached to, and extending at least partially beyond the periphery of the non-woven material layer 222 is a breathable layer 226 formed from a wicking material, such as a transparent polyurethane sheet. A suitable breathable layer 226 is a transparent polyurethane tape, about 1.0 mm in thickness, sold under the tradename IV 3000. In an alternative embodiment, a breathable layer is formed from a silicone elastomer or hydrocolloid. The polyurethane, or other breathable material, while not necessarily having as great a structural strength as the support layer, preferably has a greater capacity for absorbing moisture from the skin—i.e., wicking it away, keeping the skin relatively dry. The breathable layer may have pores 228 formed through the material, as shown in FIG. 20, to aid or provide the wicking function. The pores are sufficiently small to allow air and moisture vapor to pass through but inhibit bacteria from getting in to the skin. Alternatively, the breathable layer is air and/or moisture permeable without requiring pores, such as a hydrocolloid which allows one-way transport of moisture through the layer.

The porous layer 226 thus provides a bacteria-impermeable, water and air permeable barrier. The breathable layer 226 has a layer 230 of adhesive on its lower surface (See FIG. 19) for adhering the layer 226 to the layer 222 and to the wearer's skin in those areas where the breathable layer extends beyond the support layer. The adhesive may be a PSA, as for the first adhesive layer 224. The breathable layer is thus attached to the skin in an area 230 surrounding the point of entry to the skin of the cannula (which is the area where bacterial infection is most likely) while the support layer, with lower vapor transmission but greater strength, is attached to the skin in an area away from the cannula entry, where infection is less likely, to support the infusion set.

The infusion set is attached to the breathable layer 226 by an adhesive layer, such as a layer of double sided transfer tape 234, or other suitable attachment material, which is positioned over the support layer, as can be seen from FIG. 17. The patch thus has a multi-layer construction in use, with three, adhesively connected layers 222, 226, and 234 in a region below the infusion set 1, 100 and a single layer 226 in a region away from the infusion set, adjacent the cannula entry. The layers 226 and 222 may themselves be formed from more than one layer of material.

Release liners 236, 238, 240, such as silicone wax coated paper, are attached to the upper and lower surfaces of the patch to protect the surfaces until use. The lower release liner 240 has an aperture 242 therein which receives the cannula therethrough, and may be perforated to allow forward and rear portions 240A and 240B to be removed separately. The release liners are removed prior to use. As shown in FIG. 18, the upper release liner may be in two portions 236, 238.

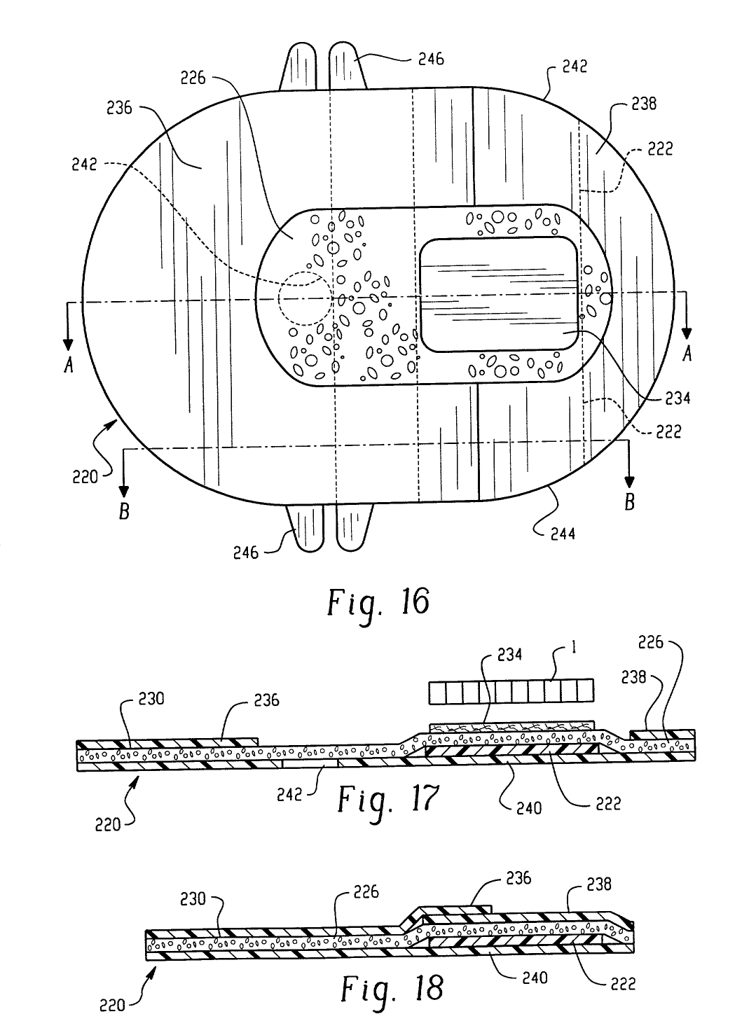

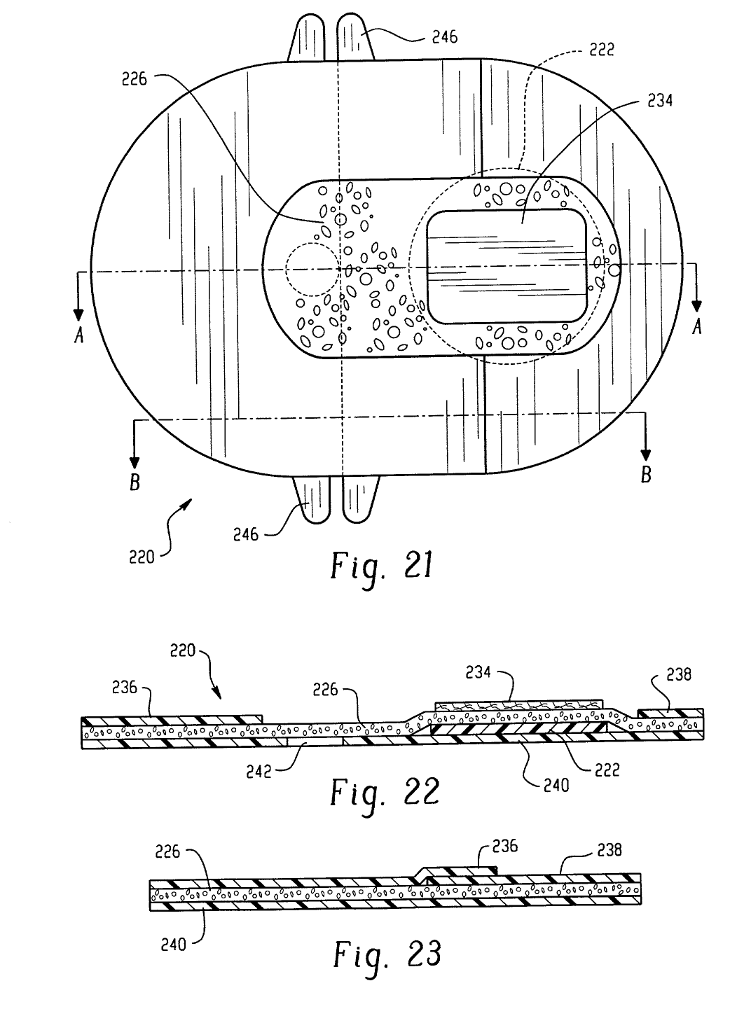

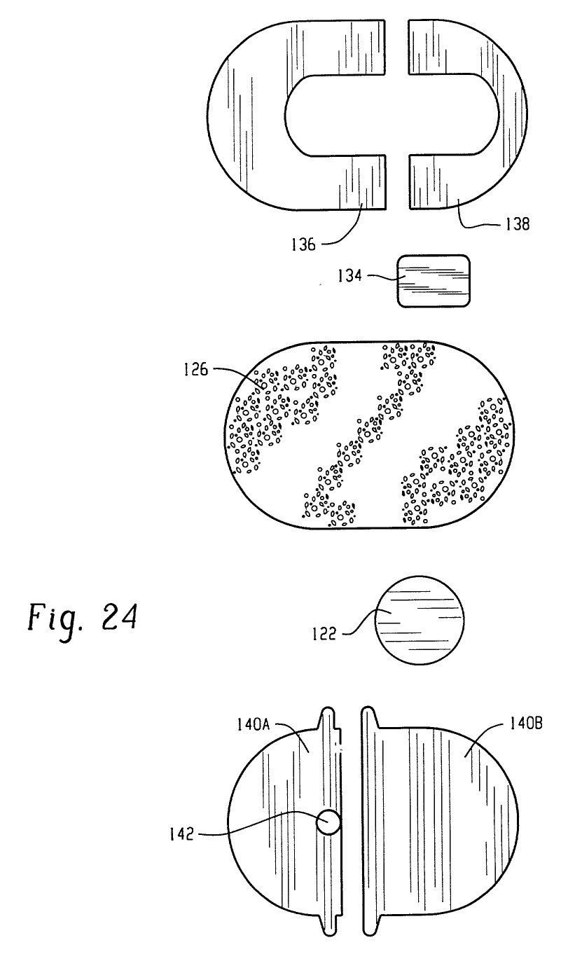

The structural layer 222 may be generally rectangular, as shown in FIGS. 16 and 20, with side edges 242, 244 extending to the periphery of the breathable layer. Or, in an alternative embodiment, shown in FIGS. 21-24, may be bounded on all sides by a portion of the breathable layer. FIG. 21 shows the structural layer 222 as circular, although other shapes which provide sufficient support to the infusion set are also contemplated. In this embodiment, the remaining layers are the same as for FIG. 16.

In both embodiments, the support layer 222 is preferably as large, or slightly larger than the infusion set so that it provides support for the entire bottom surface of both parts of the housing.

Tabs 246 on the release liners provide for easy removal of the release liners.

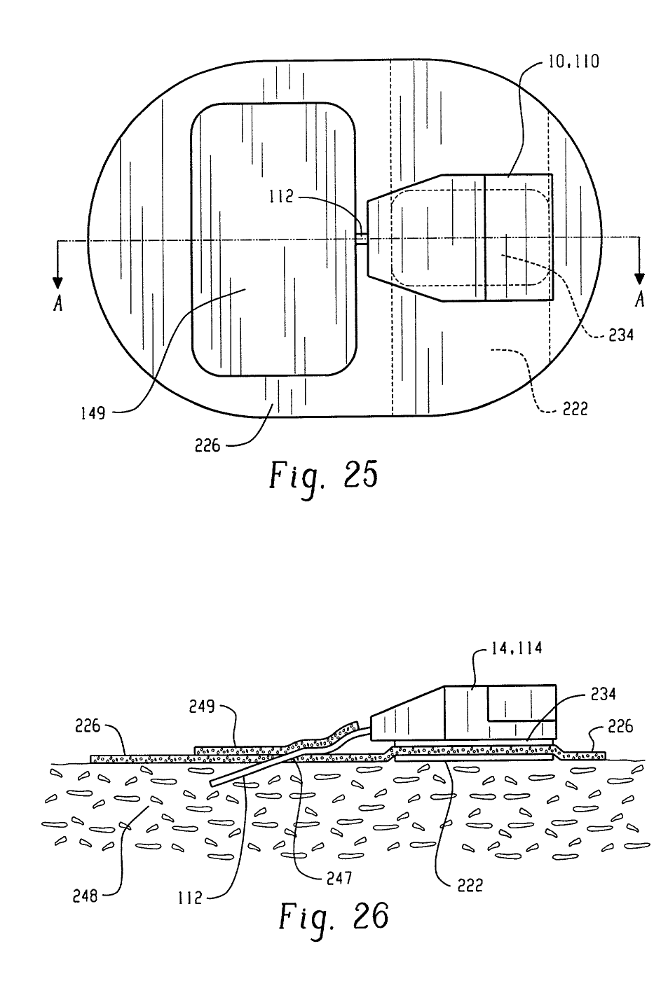

With reference now to FIGS. 25 and 26, a small hole 247 is formed in the breathable layer 226 during penetration of the cannula into the skin 248. To inhibit ingress of infectious microorganisms or dirt through the hole 247 in the layer 226 where the cannula penetrates, a protective layer or antimicrobial film 249 may be placed over the cannula in the area of the insertion region, after insertion of the cannula into the skin. The protective layer 249 may be formed from the same material as the layer 226—e.g., a polyurethane sheet about 1 mm in thickness with an adhesive PSA layer for adherence on to the layer. A release liner (not shown) may be provided on the bottom and optionally the top of the layer 249 to protect the layer until use. The two layers 226, 249 when sandwiched together, provide for air and moisture vapor to pass through but inhibit bacteria from getting in to the skin. The porous combination layer 226, 249 thus provides a bacteria-impermeable, water and air permeable barrier in a similar manner to the layer 226 alone, but provides additional protection in the area of cannula entry.

As can be seen from FIGS. 1-7, and 12, in particular, the infusion set, when assembled, has a bullet-shaped cross section, with relatively flat upper and lower surfaces, which keeps the device close to the user's skin. It can be manufactured to a small size (about 2-3 cm in length and width, or less) while nevertheless being easy to manipulate by the wearer.

It is also contemplated that the structural layer 222 be omitted and that the breathable layer 226 be of sufficient strength to support the infusion set. For example, FIG. 3 shows a tape 220′ in which the top layer 226′ is an elastomeric material, such as a silicone elastomer, which may be moisture permeable or non-moisture permeable or a hydrocolloid which is one-way permeable. In this embodiment, the layer 226′ is mechanically rather than adhesively attached to the cannula housing 14, 114, although it is also contemplated that an adhesive layer may be employed, as discussed above, either alone, or in combination with a mechanical attachment means. Suitable mechanical means for attachment include O-rings, snaps, or the like.

As shown in detail in FIG. 3A, for example, the top layer 226′ defines a cavity 250 shaped to receive a lower surface 260 of the infusion set 1, 100. The infusion set is held in the cavity by an O-ring 252 seated in a groove 254 in the cannula housing 14, 114′ and/or a corresponding groove 256 in the cavity 14. Alternatively, layer 226′ may be attached to the infusion set 1, 100 by an adhesive layer 230′, analogous to layer 230, as discussed above. A layer of an adhesive, such as a PSA, attaches the layer 226′ to the skin and is protected, prior to use by a release liner (not shown).

Suitable silicone elastomers for the layer 226′ are formed from (A) organopolysiloxane polymers having a siloxane backbone being end-blocked which may have at least two silicon-bonded groups R, wherein R denotes an olefinically unsaturated hydrocarbon substituent, an alkoxy group or a hydroxyl group, (B) a cross-linking organosilicon material preferably having at least 3 silicon-bonded reactive groups, (C) a catalyst capable of promoting the reaction between the silicon-bonded groups R of compound (A) and the silicon-bonded reactive group of compound B, although other silicone elastomers are also contemplated.

Suitable hydrocolloid adhesives are disclosed, for example, in U.S. Pat. No. 4,551,490. For example, the hydrocolloid may be formed from a mixture of mineral oil, polyisobutylene, styrene-isoprene-styrene (SIS) rubber, and antioxidant, to which a carboxymethylcellulose, cross-linked sodium carboxymethylcellulose, tackifier, and ethylene propylene rubber are added in stages.

While the adhesive patch 220 has been described with particular reference to an infusion set, it will be appreciated that other devices may be adhered to the skin with the patch. The patch may be made with appropriate dimensions for securing the desired device. It is also contemplated that the infusion set may be attached to the skin with an elastomeric rubber with adhesive applied as an alternative to the non woven tape.

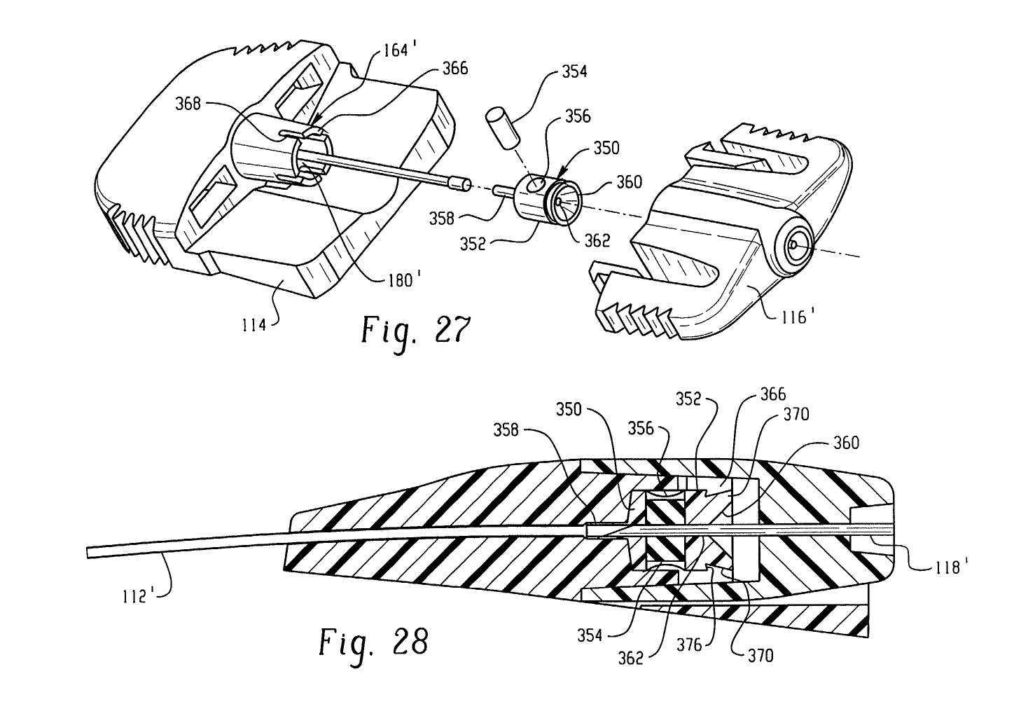

With reference now to FIGS. 27 and 28, a third alternative embodiment of an infusion set is shown, with similar parts indicated by a prime(′) and new parts given new numbers. In this embodiment, a needle housing 116′ is analogous to the needle housing of FIG. 7. A cannula housing 114′ is similar to the cannula housing 114, but differs in that a projection 164′ has a bore 180′ which is shaped to receive a seal cartridge 350 therein, in place of the needle guide and seal of FIG. 1. The seal cartridge includes a cylindrical portion 352, which receives a seal 354, similar to the seal of FIG. 7. The seal may be introduced to the cylindrical portion via a bore 356 formed in the cylindrical portion or may be inserted into the cylindrical portion during molding. A hollow tube 358 extends from one end of the cylindrical portion for connection with the catheter 112′. A conical needle guiding member 360 is defined in other end of the cylindrical portion. The seal cartridge defines a longitudinal central bore 362, generally perpendicular to the bore 356, for receiving the needle 118′ of the needle housing therethrough. The needle is long enough to pass through the seal and into the tube 358 when the two parts of the housing are connected together.

The open end of the projection 164′ defines a number of circumferentially spaced flexible tabs 366, with slits 368 therebetween. The flexible tabs 366 define inwardly projecting hooks 370. The hooks snap fit over the seal cartridge as it is inserted into the open end of the projection and seat in a circumferential locking shoulder or grove 376 defined on the seal cartridge between the cylindrical portion 352 and the needle guiding member 360.

Alternatively, the tabs may be replaced by a smooth sided projection with the seal cartridge attached to the inner surface of the projection by adhesive, welding, or other suitable means.

The invention has been described with reference to the preferred embodiment. Obviously, modifications and alterations will occur to others upon reading and understanding the preceding detailed description. It is intended that the invention be construed as including all such modifications and alterations insofar as they come within the scope of the appended claims or the equivalents thereof.

Having thus described the preferred embodiment, the invention is now claimed to be:

1. An infusion set and attachment system combination comprising:
a conformable patch for attaching the infusion set to the skin of a wearer;
the infusion set including:
a first housing portion connected to a top layer of the patch;
a second housing portion selectively interconnectable with the first housing portion for forming a fluid passageway therebetween, the second housing portion being connected with a source of a liquid to be introduced to the wearer; and
a cannula connected with the first housing portion for introducing the liquid to the wearer, the cannula passing through the patch when inserted into the wearer's skin.

2. The combination of claim 1, wherein the patch includes:
a first layer formed from a structural material.

3. The combination of claim 2, wherein the patch further includes:
a second layer formed from a moisture permeable material, through which the cannula passes.

4. The combination of claim 1, wherein the patch includes:
a layer of adhesive for attaching the patch to the wearer's skin.

5. The combination of claim 1, wherein the patch includes:
a layer of adhesive for attaching the patch to the cannula housing.

6. The combination of claim 1, wherein the patch includes a top layer which defines a cavity, the cavity being shaped to receive a lower surface of the cannula housing.

7. The combination of claim 6, further including an O-ring for selectively attaching the cannula housing to the cavity, the O-ring being received in a groove formed in at least one of the cavity and the cannula housing.

8. The combination of claim 2, wherein the first layer is formed from a non-woven material.

9. The combination of claim 8, wherein the non-woven material includes a polyester fabric.

10. The combination of claim 3, wherein the second layer is formed from a material selected from the group consisting of polyurethanes, hydrocolloids, and silicone elastomers.

11. The combination of claim 10, wherein the second layer includes a moisture permeable silicone elastomer.

12. The combination of claim 1, wherein:
the second housing portion defines a first passage which is selectively fluidly connected with the source; and
the first housing portion defines a second passage for selectively coupling with the first passage to form the fluid passageway, the first housing portion including a sealing member which is biased to a sealing position in which it seals the second passage, the sealing member being moved to a non-sealing position in which fluid flows between the first and second passages on coupling of the first and second housing portions.

13. The combination of claim 12, wherein the sealing member includes a valve, the combination further including a spring which biases the sealing member to the sealing position.

14. The combination of claim 12, wherein the first housing member includes:
a projection which defines a bore, the sealing member being movably mounted within the bore; and
a tube carried within the bore and fluidly connected with the cannula, the second passage being defined by the tube, the sealing member sealing an end of the tube in the sealing position.

15. The combination of claim 14, wherein the second housing member includes:
a generally cylindrical member fluidly connected with the source of a liquid which is received by the first housing portion bore, the generally cylindrical member defining the first passage.

16. The combination of claim 15, wherein the generally cylindrical member engages the sealing member when the first and second housing members are coupled, and moves the sealing member to the non-sealing position.

17. The combination of claim 1, wherein the patch includes a support layer and a breathable layer, the breathable layer being positioned between the support layer and the infusion set.

18. The combination of claim 17, wherein a portion of the breathable layer, extends beyond the support layer, and receives the cannula therethrough.

19. An infusion set comprising:
a first housing portion which is configured to be attached to a wearer's skin, the first housing portion defining a first fluid passage; and
a second housing portion which is selectively connectable with the first housing portion which defines a second fluid passage;
sealing means which is biased to a sealing position to seal one of the first and second passages when the first and second housing portions are disconnected, the sealing means being moved to a non-sealing position during interconnection of the first and second housing portions such that the first and second passages to form a fluid flowpath whereby a liquid flows from the first housing portion to the second housing portion; and
connection means for selectively interconnecting the first and second housing portions.

20. The infusion set of claim 19, wherein the connection means include:
flexible arms on one of the first and second housing portions, the arms defining hooks; and
slots on the other of the first and second housing portions which receive the hooks.

21. A method for supplying a liquid to a person including:
attaching a patch to the skin of the person;
attaching a first housing portion to the patch;
inserting a cannula of the first housing portion through the patch and into the person's skin;
selectively coupling a second housing portion with the first housing portion to form a fluid flowpath therebetween which is fluidly connected with the cannula; and
feeding the liquid to the first housing portion, the liquid flowing along the fluid flowpath and into the cannula.
